(19) United States
(12) Patent Application Publication
(10) Pub. No.: US 2018/0170839 A1
(43) Pub. Date: Jun. 21, 2018

(54) Recovery of Hydrogen and Ethylene from Fluid Catalytic Cracking Refinery Off Gas

(71) Applicant: Janus Technology Solutions, LLC, Spring, TX (US)

(72) Inventors: Sabah A. Kurukchi, Spring, TX (US); Joseph M. Gondolfe, Magnolia, TX (US)

(73) Assignee: Janus Technology Solutions, LLC, Spring, TX (US)

(21) Appl. No.: 15/843,776

(22) Filed: Dec. 15, 2017

Related U.S. Application Data
(60) Provisional application No. 62/435,183, filed on Dec. 16, 2016.

(51) Int. Cl. C07C 7/11; C07C 6/04; C07C 2/64; C07C 2/06; C07C 4/06; C07C 17/02; C07C 7/04; B01D 53/14; B01D 53/047; B01D 53/18; B01D 3/14

(52) U.S. Cl. CPC C07C 7/11; B01D 2257/7022; C07C 2/64; C07C 2/06; C07C 4/06; C07C 17/02; C07C 7/04; B01D 53/1487; B01D 53/047; B01D 53/1425; B01D 53/18; B01D 3/143; B01D 2252/205; B01D 2257/108; C07C 6/04

(57) ABSTRACT

A method comprising: introducing a refinery off gas stream into an oil absorber wherein the refinery off gas stream comprises H₂, N₂, O₂, methane, ethane, ethylene, propane, propylene, and C₄+; introducing a solvent into the oil absorber; counter-currently contacting the refinery off gas stream and the solvent in the oil absorber; generating an absorber overhead stream comprising H₂, N₂, O₂, and methane; generating an absorber bottoms stream comprising the solvent wherein ethane, ethylene, propane, propylene, and C₄+ are dissolved in the solvent; introducing the absorber bottoms stream into a solvent regenerator and generating an overhead stream comprising ethane, ethylene, propane, propylene, and C₄+; and introducing the overhead stream into a C₂-C₃ splitter that generates a dilute ethylene product stream and a bottoms product stream, wherein the dilute ethylene product stream comprises ethylene and ethane, and wherein the bottoms product stream comprises propane, propylene, and C₄+.

### BACKGROUND

In petroleum refining operations, hydrogen and ethylene contained within Refinery Off-Gas (ROG) from a Fluidized Catalytic Cracker Unit (FCCU) may be routed to the internal fuel gas system of the refinery. With the growing recognition of gross margins above fuel value for both hydrogen and ethylene, base petrochemical producers of lower olefins may opt to co-process ROG to separate hydrogen and ethylene. Integration of ROG with base petrochemical production may afford the opportunity to utilize available existing unit operations such as cryogenic recovery of hydrogen and purification of ethylene together with the cracked gases already produced from steam cracking. This integration option may only be practical when the base petrochemical plant and the FCCU refinery are adjacent or at close proximity to each other.

A particular challenge of integrating ROG with base chemical production is contaminants found in ROG which are incompatible with other processes whether related to the refinery or petrochemical sector. In particular, NOx, oxygen and N₂ in the typical concentrations produced from the FCCU refinery may deposit explosive compounds, typically formed as nitroso gums, when exposed to cryogenic downstream units or most other units which cannot tolerate such contaminants.

### BRIEF DESCRIPTION OF THE DRAWINGS

These drawings illustrate certain aspects of some of the embodiments of the present invention, and should not be used to limit or define this disclosure.

FIG. 1 is a schematic illustration of a fluidized catalytic cracking unit.

FIG. 2 is a schematic illustration of a system for removing contaminants and recovering hydrogen and ethylene from an ROG feed.

FIG. 3 is a schematic illustration of a system for removing contaminants and recovering hydrogen and ethylene from an ROG feed with cost effective heat integration scheme.

FIG. 4 is a schematic illustration of additional processes for a dilute ethylene product stream.

### DETAILED DESCRIPTION

The present disclosure may generally relate to refining methods and unit operations associated with the Hydrocarbon Processing Industry (HPI). Provided herein are methods that, in some embodiments, may include separation of ethylene and heavier hydrocarbons from hydrogen and methane by an Oil Absorber from a refinery off gas stream from a fluidized catalytic cracker unit. In some embodiments, the hydrogen and methane may be further separated by a pressure swing adsorption (PSA) system or other equivalent unit operation known in the art. In some embodiments, ethane and ethylene may be separated from other products by distillation.

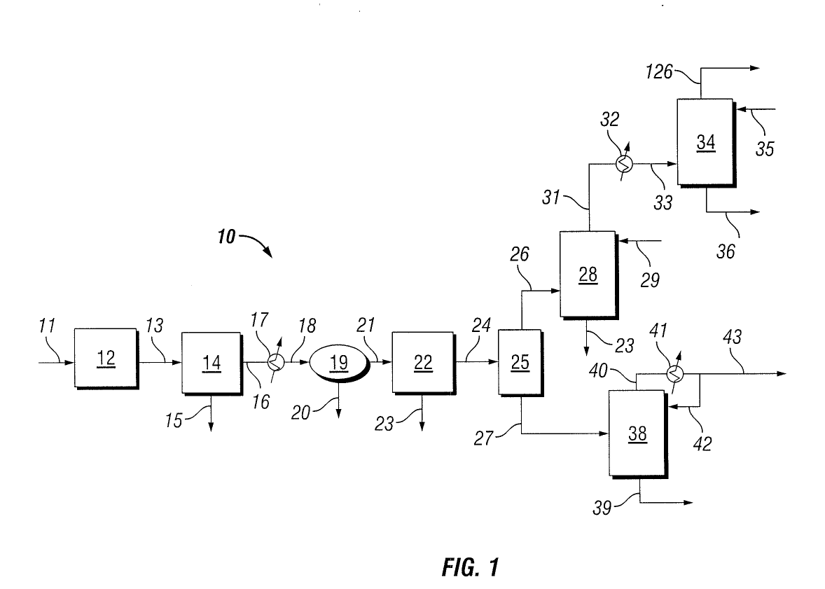

An FCCU may crack long chain hydrocarbons with relatively high boiling points of about 300° C. to about 450° C. to lower molecular weight hydrocarbons. The feedstock for an FCCU may be hydrotreated distillate from an atmospheric and/or vacuum distillation column or any other suitable feedstocks well known in the art. An example of a FCCU unit with its corresponding gas plant is illustrated in FIG. 1.

In the illustrated embodiment within FIG. 1, the FCC heavy feedstock 11 may be fed to the FCC reactor 12 where it may be cracked to hydrogen, light hydrocarbons, acid gases, and various contaminants. Light hydrocarbons may include methane, ethylene, ethane, propylene, propane, C4 olefin/saturate, gasoline, naphtha, light cycle oil (LCO) and clarified oil. The FCC heavy feedstock 11 may contain heavy atmospheric gas oil (HAGO) or vacuum gas oil (HVGO). The HVGO may include the portion of the crude oil with an initial boiling point of 340° C. or higher at atmospheric pressure and an average molecular weight ranging from about 200 to 600 or higher. The reactor effluents 13 may be cooled in a Main Fractionator 14 (e.g., a multi-section column with pump arounds, which may be equipped with means for withdrawing a naphtha, kerosene, light cycle oil and heavy cycle oil). The total Main Fractionator overhead vapor may comprise the raw gasoline product and lighter hydrocarbons together with steam and inert gas from the reactor system. For example, the overhead vapor may comprise on average by mole 25% H₂O, 1% N₂, 0.2% CO₂, 0.01% H₂S, 8% C1, 7% ethylene, 2.5% ethane, 19% propylene, 15% C4's, 3% C5's and balance C6⁺. The main fractionator bottoms product 15 may comprise relatively heavier hydrocarbons, such as heavy cycle oil (HCO) and slurry oil which are recycled back to the reactor for further cracking; one side product from the main fractionator is the light cycle oil (LCO) whose True Boiling Point (TBP) is typically 140° C. IBP to 370° C. FBP and largely mono/di/tri/polynuclear aromatics of 75% by volume. The Main Fractionator reflux 16, a portion of the steam and a small amount of distillate may be condensed in the Main Fractionator Overhead Condenser 17 and passed to overhead accumulator 19 as stream 18 where sour condensate water 20 with dissolved acids, and part of the alcohols, ammonia and HCN is separated, the gas 21 from accumulator 19 may then be compressed in multistage compressor with interstage cooling unit 22, wherein water condensate 23 may be separated that comprises dissolved oxygenates, nitriles and other contaminants. The gas 21 from accumulator 19 may comprise on average by mole 6.4% H₂O, 1.8% N₂, 0.4% CO₂, 0.015% H₂S, 10.6% C1, 14% C2's, 29% C3's, 20% C4's, 5% C5's and balance C6⁺.

In the illustrated embodiment within FIG. 1, the gas 21 may continue from compressor with interstage cooling unit 22 to Absorber/Stripper 25. As illustrated, the Absorber/Stripper 25 may recover C₃⁺ hydrocarbons in the absorber bottoms 27, and the Absorber/Stripper 25 may strip the C₂'s hydrocarbons and lighter components from the high pressure liquid to form a lights stream 26. Lights stream 26 may comprise on average by mole 95% C2's & Lighter and 5% C3's & Heavier.

The Sponge Absorber 28 may recover most of the C₃+ hydrocarbons entrained in the vapors (e.g., lights stream 26) leaving the Absorber/Stripper 25. In some embodiments, lean oil from the Main Fractionator 14 may be the absorption medium used in the Sponge Absorber 28. Sponge Absorber overhead 31 may be cooled in the off gas Cooler 32 before the cooled stream 33 may be fed into the amine absorber 34 with lean amine stream 35 for removal of CO₂ and H₂S from the refinery off gas (ROG) 33 and producing an amine treated ROG product 126, which may comprise ethylene and lighter components. Lean amine stream 35 may comprise, for example, methyldiethanolamine (MDEA) about 30 to 45wt % in water. A rich amine stream 36 may be removed from the amine absorber 34 and routed to the amine regenerator. The rich amine stream 36 may comprise MDEA, absorbed acid gases and hydrocarbons.

The C₃⁺ liquid bottoms stream 27 may be fractionated in a debutanizer (DC4) tower 38 to provide a C₅⁺ liquid gasoline product 39 and LPG product 40. The DC4 overhead gas (e.g., LPG product 40) may be condensed in DC4 condenser 41 providing reflux 42 to the DC4 tower 38 and C3/C4 LPG stream 43.

Table 1 lists an example of an ROG composition. Although the compositions listed in Table 1 may be typical, one of ordinary skill would understand that concentration of each component may vary depending on the source of feedstock and catalysts are used in the FCCU. Additionally, one of ordinary skill in the art will understand that the typical minimum and maximum values given may also vary and any change would not deviate from the scope of this disclosure.

TABLE 1
Typical FCCU-ROG Composition

| | | | Minimum | Maximum |
|---|---|---|---|---|
| Component | wt % | mol % | wt % | wt % |
| H₂O | 1.27 | 1.196 | 0.5 | 1.5 |
| Oxygen | 0.57 | 0.3 | 0.1 | 0.6 |
| Nitrogen | 16.88 | 10.184 | 3.0 | 11.0 |
| CO | 0.99 | 0.599 | 0.1 | 0.6 |
| CO₂ | 1.82 | 0.699 | 0.03 | 2.0 |
| H₂S | 0.02 | 0.01 | 0.01 | 0.03 |
| H₂ | 4.01 | 33.647 | 4.0 | 10.0 |
| Methane | 25.39 | 26.748 | 20.0 | 30.0 |
| Acetylene | 0.015 | 0.01 | 0.004 | 0.02 |
| Ethylene | 19.72 | 11.881 | 15.0 | 20.0 |
| Ethane | 20.92 | 11.761 | 15.0 | 25.0 |
| Propylene | 4.52 | 1.817 | 0.9 | 5.0 |
| Propane | 0.96 | 0.369 | 0.9 | 5.0 |
| 1-Butene | 0.86 | 0.26 | 0.1 | 1.0 |
| i-Butane | 0.41 | 0.12 | 0.1 | 0.5 |
| Butane | 0.45 | 0.13 | 0.1 | 0.5 |
| Pentane | 1.19 | 0.28 | 0.2 | 1.2 |
| Total | 100 | 100 | | |
| Total MWT | 16.902 | 16.902 | | |

In some refineries, ROG may be treated in a catalytic hydrogenation unit to remove some contaminants. In particular, O₂ and NOx may be converted to water and ammonia respectively with catalytic hydrogenation. In some refineries, two catalyst formulations may be used to serve this purpose. The first may be sulfided copper and the second may be sulfided nickel. One potential disadvantage of bulk phase catalytic hydrogenation is the capital cost of the catalysts and associated equipment and overall operational costs of the system. Another potential disadvantage to the refiner may be that the treated ROG will have to be transported to the petrochemical production process side of the refinery to the catalytic hydrogenation unit thereby limiting the practical distance between the FCCU and the ROG Unit itself.

While not shown, the amine treated ROG 126, in some embodiments, may be compressed and washed in a caustic tower with a caustic solution to further remove both CO₂ and H₂S, each to less than approximately 1 mppm.

Acetylene may be a contaminant in some ROG streams. Acetylene present in the ROG steam may contaminate the final dilute ethylene product which may be problematic for some end users or uses of the dilute ethylene product. In such examples, it may be required to remove acetylene from the ROG stream upstream of the oil absorption column. If required, the ROG stream containing the acetylene may be selectively hydrogenated within an adiabatic acetylene hydrogenation reactor to produce ethylene as the major product and ethane as the minor product.

Embodiments of the systems described herein may recover dilute ethylene (ethylene-ethane) from a gaseous mixture containing large proportions of methane (e.g. >20 mol %), nitrogen (e.g. >5 mol %) and hydrogen (e.g. >25 mol %) by the use of a reboiled Oil Absorber fed with lean C6-C8 absorption oil to recover C2+ components in the ROG feed. The absorption oil, often referred to as “solvent”, may have low volatility compared to feed components so it may remain in liquid phase during absorption. Furthermore, in some embodiments, the solvent should be readily separable in a stripper from the absorbed components. In some embodiments, the K value (mole of gas in liquid phase/mole of gas in vapor phase) should be high for ethylene and ethane for the solvent to be viable. A higher the K value of ethylene and ethane for any selected solvent may result in a smaller amount of recirculating solvent required for the same mass of ROG feed to the Oil Absorber. Some examples of suitable solvents may include, but are not limited to, cyclohexene, cyclohexane, hexane, hexene, heptane, octane, gasoline, kerosene, aromatic distillate, and combinations thereof. Table 2 lists some exemplary solvents and their associated K-values for ethylene and ethane. One of ordinary skill will understand that the K-values of each component in a mixture will be dependent on the composition of the mixture. One of ordinary skill in the art with the benefit of this disclosure should be able to select an appropriate solvent for a given ROG feed.

TABLE 2
Solvents and K-Values

| SOLVENTS | K-ETHYLENE | K-ETHANE |
|---|---|---|
| Cyclohexane | 2.2 | 3.3 |
| n-Hexane | 1.1 | 2.2 |
| Kerosene | 1.5 | 2.1 |

Embodiments of the reboiled Oil Absorber may remove substantially all of the ethylene and heavier hydrocarbon components of the ROG from the gaseous phase, leaving methane, nitrogen and hydrogen to be recovered by pressure swing absorption or other methods known in the art. By removing the light contaminants such as methane and hydrogen, the product dilute ethylene may be substantially free of these components, for example, less than 1 mppm (molar part per million). One of ordinary skill in the art will understand that methane and hydrogen contamination in ethylene streams may cause adverse problems in processes that utilize ethylene and therefore a product dilute ethylene stream with methane and hydrogen contamination may be less valuable or unusable.

Furthermore, situating the reboiled Oil Absorber before the processes to separate dilute ethylene, in some embodiments, may reduce the unavoidable material loss of ethylene. Conventional processes to remove the aforementioned contaminants by distillation may have large losses of ethylene compared to the present process with the reboiled Oil Absorber. As a value-added feature, this scheme may remove essentially all the contained O₂ and NOx from the ROG with the overhead hydrogen, nitrogen and methane stream; thus, the recovered dilute ethylene product may be substantially free of O₂ and NOx, for example, below 1 mppm or in some examples, blow 1 mppb (molar part per billion), or in some examples 1 mppb. The absorber overhead gas may be separated in a PSA H₂ unit or other suitable separation unit into a hydrogen rich stream and a methane rich stream.

In some embodiments, the rich oil stream from the reboiled Oil Absorber may be distilled in a Solvent Regenerator to an overhead containing the C2-C4 components in the ROG feed and the Solvent Regenerator bottoms is the lean oil stream may be cooled and chilled, then recycled back to the upstream reboiled Oil Absorber.

In some embodiments, the overhead C2-C4 stream of the Solvent Regenerator may be further distilled in a C2-C3 Splitter to an overhead containing the ethylene-ethane (dilute ethylene) product stream and a C2-C3 Splitter bottoms containing the C3-C4 components of the ROG feed, for example which may be sent for processing in the FCCU Debutanizer Tower for increased recovery of the C3's and C4's components.

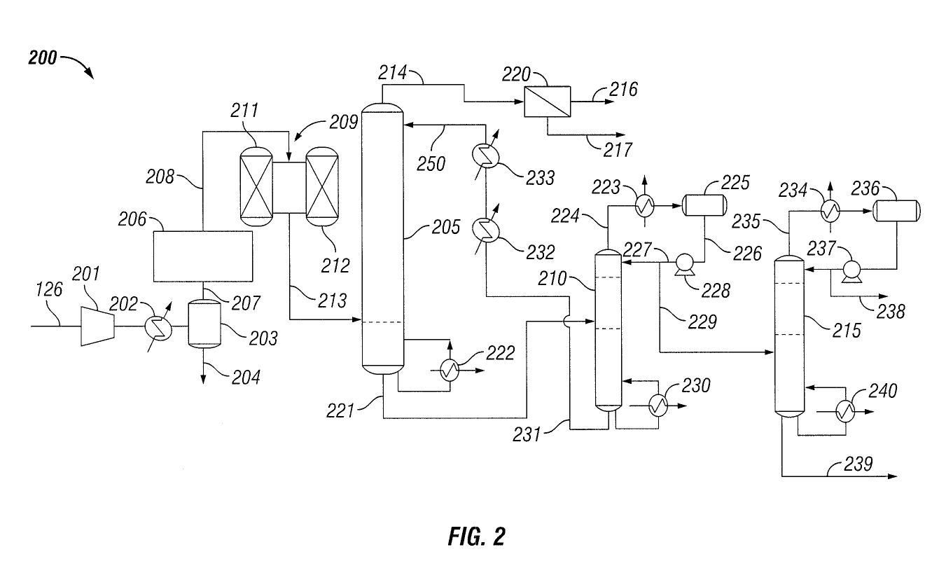

Examples of the methods of removing contaminants and separating products from an ROG stream will be described in more detail with reference to FIG. 2. A system 200 for removing contaminants and separating products from an FCC ROG is illustrated in accordance with present embodiments. System 200 may comprise reboiled Oil Absorber 205, Solvent Regenerator 210, and a C2-C3 Splitter 215.

ROG stream 126 from fluidized catalytic cracker 12 (illustrated in FIG. 1) may be compressed in compressor 201from approximately 10 to 12 barg (bar gauge) and approximately 40 to 45° C. to approximately 30 to 35 barg and cooled by cooling water exchanger 202 to maintain the same temperature, and subsequently passed to phase separator drum 203. Phase separator drum 203 may knock out condensed water and condensed hydrocarbons. Oily sour water 204 may be discharged to the sour water drains. Compressed gas 207 from phase separator drum 203 may be passed to a contaminant removal unit 206.

Contaminant removal unit 206 may comprise several units depending on the downstream requirements. Examples of contaminant removal units were previously discussed. In some examples, contaminant removal unit 206 may comprise a caustic tower. A caustic tower may treat compressed gas stream 207 and may reduce CO₂ and H₂S concentrations, for example, to approximately 1 mppm or lower. In some embodiments, a caustic tower may use about 10 to 12 wt % NaOH in a standard design caustic tower with two or three stage caustic sections follows by water wash section. In some examples, it may be required to reduce acetylene content to approximately 1 to 5 mppm in the dilute ethylene product. In such examples, compressed gas 207 may be passed into an acetylene hydrogenation unit. Examples of an acetylene hydrogenation unit may comprise a reactor and a Pd based catalyst. Commercial examples of the catalyst may be supplied by Chevron-Phillips Chemicals or Clariant (Sud Chemie). One of ordinary skill in the art with the benefit of this disclosure should be able to select an appropriate contaminant removal unit for a particular application.

In the illustrated embodiment, clean ROG stream 208 from contaminant removal unit 206 may be passed to molecular sieve driers or any other suitable equipment for water removal. In some embodiments, mole sieve driers 209 may reduce water content of clean ROG stream 208, for example, down to less than about 1 mppm. Mole sieve driers 209 may comprise any suitable molecular sieve. In particular, 3A mole sieve may be used. Removal of water may be necessary to ensure the downstream process streams do not freeze when exposed or contacted with cold process streams. As illustrated, the mole sieve driers may comprise two or more interconnected beds 211 and 212. One bed may be operating while the other may be in regeneration or standby mode. The drier beds may be regenerated by dry fuel gas from the PSA H₂ unit 220 and then returned to the plant central fuel gas drum or may be dried by other means such as plant nitrogen.

Dry ROG gas 213 may be at any suitable temperature and pressure, including, but not limited to, approximately 29 to 34 barg and approximately 40 to 50° C. Dry ROG gas 213 may be fed to reboiled Oil Absorber tower 205, which may be a packed or trayed tower. Reboiler 222 may be operated at any suitable temperature, including, but not limited to, approximately 130° C. to 140° C.; the reboiled Oil Absorber tower 205 may be fed at the top with lean absorption oil stream 250 that may be cooled to any suitable temperature, including, but not limited to, approximately −35° C. to −40° C. In the illustrated embodiment, the lean absorption oil stream 250 may be counter-currently contacted with dry ROG gas 213 that may flow up from the bottom of the reboiled Oil Absorber tower 205. Light components such as methane, nitrogen, NOx, CO, and hydrogen present in the ROG stream may have extremely low solubility in the absorption oil. The light components may exit the top of the reboiled Oil Absorber tower 205in light stream 214. Light steam 214 may be sent for further processing in a PSA H₂ unit 220 for separation into hydrogen rich stream 216 and methane rich stream 217 which may be compressed and sent to the fuel gas drum.

Rich oil solvent 221 containing the absorbed ethylene, ethane, C3's, and C4+ components of feed ROG may exit the bottom of reboiled Oil Absorber 205. One of ordinary skill in the art will understand that any amount of ethylene may be recovered from dry ROG gas 213 in reboiled Oil Absorber 205. The amount of ethylene recovered may be dependent on many factors, including, but not limited to, mass flow rate of dry ROG gas 213, mass flow rate of lean absorption oil stream 250, and operating conditions of reboiled Oil Absorber tower 205. The choice of percentage recovery may be based on many factors as one of ordinary skill in the art will understand. In some examples, reboiled Oil Absorber 205 may be designed to recover approximately 75 to 80% or greater of the ethylene present in dry ROG gas 213. In some embodiments, the lean absorption oil stream 250 rate may be approximately 2.5 to 4.5 times the mass flow rate of the ROG feed stream to the absorber depending on the K-values of the solvent and solute as previously discussed.

In the illustrated embodiment, rich oil solvent 221 leaving reboiled Oil Absorber 205 may be pumped and fed to Solvent Regenerator 210. Solvent Regenerator 210 may comprise a trayed or packed tower, and may be operated at any suitable pressure, including, but not limited to, approximately 30-35 barg. In some embodiments, refrigerated reflux condenser 223 may be operated at a temperature low enough such that the overhead stream 224 is condensed and collected in Solvent Regenerator reflux drum 225. The liquid C2-C4 stream 226 may be returned to Solvent Regenerator 210 as reflux 227 through pump 228. Additionally, a portion of liquid C2-C4 stream 226 may be withdrawn under flow control as liquid C2-C4 product stream 229 and send to C2-C3 Splitter tower 215.

Solvent Regenerator reboiler 230 may be operated at any suitable temperature, including, but not limited to, approximately 260° C. to about 280° C. The stripping rate may be adjusted by controlling the heat rate to the reboiler 230. In some embodiments, the reboiler heat rate may be adjusted such that the methane concentration in the bottoms stream is less than 0.1 mppm. A bottoms stream 231 may be withdrawn from Solvent Regenerator 230 and cooled through heat exchanger 232, for example, to atmospheric temperature, and chilled in the refrigerated cooler 233 to any suitable temperature, for example, approximately −30 to −35° C., before recycling to the reboiled Oil Absorber 205.

C2-C3 Splitter 215 may comprise a trayed or packed tower, which may be operated at any suitable pressure, including, but not limited to, approximately 29-34 barg. In some embodiments, refrigerated reflux condenser 234 may be operated at a temperature such that the overhead stream 235 may be condensed. In the illustrated embodiment, the liquid ethylene-ethane stream may be collected in the regenerator reflux drum 236 returned to the column via pump 237 as reflux. A portion of the condensed overhead stream may be withdrawn as product dilute ethylene-ethane stream 238 may be withdrawn and sent to the dilute ethylene product storage facility or other further processing steps.

In some embodiments, C2-C3 Splitter 215 may be designed for the product dilute ethylene-ethane stream 238 to comprise a propylene content less than about 1.0 mppm; and the C2-C3 Splitter 215 bottoms stream 239 to comprise the C3's-C4's with ethane content to be less than approximately 5.0 mppm.

The C2-C3 Splitter reboiler 240 may be operated at any suitable temperature, including about 70° C. to about 90° C. Bottoms stream 239 drawn from C2-C3 Splitter 215 may comprise substantially all the C3's-C4+in Clean ROG stream 208. For example, about 99.9 mole % to about 99.999 mole % of the C3's-C4+. Bottoms stream 239 may be sent for processing in the FCC unit debutanizer tower for recovery.

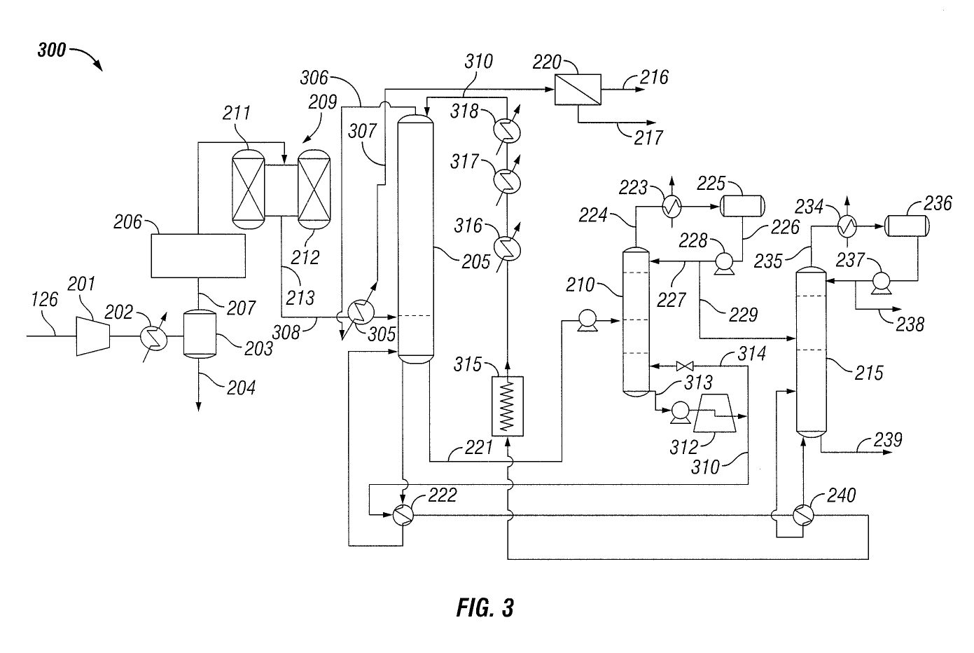

An illustrative example of the foregoing method is described by the following overall material balance within Table 3. The streams IDs correspond to the streams of FIG. 3. The data was generated using a process simulator.

TABLE 3
Illustrative Material Balance

| STREAM ID | 126 | 308 | 306 | 310 | 221 | 229 | 238 | 239 |
|---|---|---|---|---|---|---|---|---|
| NAME | ROG | ROG | ABS | Recir | ABS | Splitter | DIL | C3-C4 |
| | FD | to Oil | OVHD | ABS OIL | BTMS | FD | ETHLN | PRDCT |
| | | ABS | | | | | | |
| PHASE | WET | DRY | DRY | DRY | DRY | DRY | DRY | DRY |
| | VAP | VAP | VAP | LIQ | LIQ | LIQ | LIQ | LIQ |
| RATES, KG/HR | | | | | | | | |
| 1 H2O | 160.75 | 0.00 | 0.00 | 0.00 | 0.00 | 0.00 | 0.00 | 0.00 |
| 2 O2 | 114.54 | 114.54 | 114.54 | 0.00 | 0.00 | 0.00 | 0.00 | 0.00 |
| 3 N2 | 3391.87 | 3391.87 | 3391.86 | 0.00 | 0.00 | 0.00 | 0.00 | 0.00 |
| 4 CO | 198.93 | 198.93 | 198.93 | 0.00 | 0.00 | 0.00 | 0.00 | 0.00 |
| 5 CO2 | 365.71 | 0.00 | 0.00 | 0.00 | 0.00 | 0.00 | 0.00 | 0.00 |
| 6 H2S | 4.02 | 0.00 | 0.00 | 0.00 | 0.00 | 0.00 | 0.00 | 0.00 |
| 7 H2 | 805.77 | 805.77 | 805.77 | 0.00 | 0.00 | 0.00 | 0.00 | 0.00 |
| 8 METHANE | 5101.88 | 5101.88 | 5101.55 | 0.00 | 0.31 | 0.31 | 0.31 | 0.00 |
| 9 ETHYLENE | 3962.54 | 3962.54 | 1201.24 | 0.02 | 2761.31 | 2761.29 | 2761.29 | 0.00 |
| 10 ETHANE | 4203.67 | 4203.67 | 20.85 | 0.13 | 4182.83 | 4182.70 | 4182.23 | 0.47 |
| 11 PROPYLENE | 908.25 | 908.25 | 0.00 | 1.78 | 908.25 | 906.41 | 0.35 | 906.06 |
| 12 PROPANE | 196.92 | 196.92 | 0.00 | 0.62 | 196.92 | 196.28 | 0.00 | 196.27 |
| 13 1BUTENE | 172.81 | 172.81 | 0.00 | 24.36 | 172.81 | 147.61 | 0.00 | 147.61 |
| 14 IBUTANE | 82.39 | 82.39 | 0.00 | 6.69 | 82.39 | 75.47 | 0.00 | 75.47 |
| 15 BUTANE | 90.42 | 90.42 | 0.00 | 19.52 | 90.42 | 70.23 | 0.00 | 70.23 |
| 16 ACETYLEN | 0.40 | 0.40 | 0.00 | 0.00 | 0.40 | 0.40 | 0.40 | 0.00 |
| 17 PENTANE | 239.12 | 239.12 | 2.97 | 983.05 | 1036.15 | 19.39 | 0.00 | 19.39 |
| 18 ABS OIL | 0.00 | 0.00 | 28.65 | 78963.83 | 79171.50 | 0.02 | 0.00 | 0.02 |
| TOTAL, KG/HR | 20000.00 | 19469.52 | 10866.36 | 80000.00 | 88603.30 | 8360.11 | 6944.58 | 1415.53 |
| TEMP, C. | 44.00 | 4.60 | −25.40 | −35.00 | 139.82 | 8.73 | −1.78 | 80.47 |
| PRES, BAR(G) | 10.70 | 33.46 | 32.00 | 36.00 | 32.50 | 31.80 | 29.00 | 30.30 |
| MWT | 16.90 | 16.70 | 12.16 | 81.98 | 71.01 | 31.08 | 29.23 | 45.07 |
| WT FR VAP | 1.00 | 1.00 | 1.00 | 0.00 | 0.00 | 0.00 | 0.00 | 0.00 |
| WT FR LIQ | 0.00 | 0.00 | 0.00 | 1.00 | 1.00 | 1.00 | 1.00 | 1.00 |

With reference to FIG. 3, a heat integrated system 300 for removing contaminants and separating products from an FCC ROG is illustrated in accordance with present embodiments ROG stream from fluidized catalytic cracker 110 (e.g., illustrated in FIG. 1) may be treated by an amine absorber (not shown) before entering compressor 201. ROG stream 126 may enter compressor 201 at any suitable pressure and temperature, including, but not limited to, approximately 10 to 12 barg (bar gauge) and approximately 40 to 45° C. ROG stream 126 may be compressed in compressor 201 to any suitable pressure, including, but not limited to, approximately 30 to 35 barg and cooled by cooling water exchanger 202, for example, to a temperature of approximately 40 to 45° C., and subsequently passed to phase separator drum 203. Phase separator drum 203 may knock out condensed water and condensed hydrocarbons. Oily sour water 204 may be discharged to the sour water drains. Compressed gas 207 from phase separator drum 203 may be passed to a contaminant removal unit 206.

Contaminant removal unit 206 may comprise several units depending on the downstream requirements. Examples of contaminant removal units were previously discussed. In some examples, contaminant removal unit 206 may comprise a caustic tower. A caustic tower may treat compressed gas stream 207 and may reduce CO₂ and H₂S concentrations, for example, to approximately 1 mppm or lower. In some embodiments, a caustic tower may use about 10 to 12 wt % NaOH in a standard design caustic tower with two or three stage caustic sections follows by water wash section. In some examples, it may be required to reduce acetylene content, for example, to approximately 1 to 5 mppm, in the dilute ethylene product. In such examples, compressed gas 207 may be passed into an acetylene hydrogenation unit. An acetylene hydrogenation unit may comprise a reactor and a Pd based catalyst. Commercial examples of the catalyst may be supplied by Chevron-Phillips Chemicals or Clariant (Sud Chemie). One of ordinary skill in the art with the benefit of this disclosure should be able to select an appropriate contaminant removal unit for a particular application.

In the illustrated embodiment, clean ROG stream 208 from contaminant removal unit 206 may be passed to molecular sieve driers or any other suitable equipment for water removal. Mole sieve driers 209 may reduce water content of clean ROG stream 208, for example, down to less than about 1 mppm. Mole sieve driers 209 may comprise any suitable molecular sieve. In some embodiments, 3A mole sieve may be used. Removal of water may be necessary to ensure the downstream process streams do not freeze when exposed or contacted with cold process streams. In the illustrated embodiment, the mole sieve driers may comprise two or more interconnected beds 211 and 212. One bed may be operating while the other may be in regeneration or standby mode. The drier beds may be regenerated by dry fuel gas from the PSA H₂ unit 220 and then returned to the plant central fuel gas drum or may be dried by other means such as plant nitrogen.

Dry ROG gas 213 may be at any suitable pressure and temperature, including a pressure in a range of about 29 to about 34 barg and a range in temperature between 40 to 50° C. Dry ROG gas 213 may be cooled in heat exchanger 305 by contact with the cold overhead stream 306 from reboiled Oil Absorber 205. Heat exchanger 305 may bring warm overhead stream 307 to any suitable temperature, for example, to approximately 41° C. Cooled dry ROG gas 308 may be fed to the reboiled Oil Absorber 205, which may be a packed or trayed tower. Reboiler 222 may be operated at any suitable temperature, including, approximately 130° C. to 145° C. Reboiled Oil Absorber 205 may be fed at the top with lean absorption oil stream 310 that may be cooled to any suitable temperature, for example, a temperature of approximately −35° C. to −40° C. In the illustrated embodiment, the lean absorption oil stream 310 may be counter-currently contacted with dry ROG gas 308 that may flow up from the bottom of the reboiled Oil Absorber 205. Light components such as methane, nitrogen, NOx, CO, and hydrogen present in dry ROG gas 308 may have extremely low solubility in the absorption oil. The light components may exit the top of the reboiled Oil Absorber 205 in cold overhead stream 306. Cold overhead stream 306 may be heated in exchanger 305, for example, to about 38° C. to about 41° C. Warm overhead stream 307 may be sent for further processing in a PSA H₂ unit 220 for separation into hydrogen rich stream 216 and methane rich stream 217 which may be compressed and sent to the fuel gas drum.

Rich oil solvent 221 containing the absorbed ethylene, ethane, C3's, and C4+ components of feed ROG may exit the bottom of reboiled Oil Absorber 205. One of ordinary skill in the art will understand that any amount of ethylene may be recovered from cooled dry ROG gas 308 in reboiled Oil Absorber 205. The amount of ethylene recovered may be dependent on many factors, including, but not limited to, mass flow rate of cooled dry ROG gas 308, mass flow rate of lean absorption oil stream 310, and operating conditions of reboiled Oil Absorber tower 205. The choice of percentage recovery may be based on many factors as one of ordinary skill in the art will understand. In some examples, reboiled Oil Absorber 205 may be designed to recover approximately 75 to 80% or greater of the ethylene present in cooled dry ROG gas 308. In some embodiments, the lean absorption oil stream 310 rate may be approximately 2.5 to 4.5 times the mass flow rate of the ROG feed stream to the absorber depending on the K-values of the solvent and solute as previously discussed. The stripping rate may be adjusted by controlling the heat rate to the reboiler 230.

Rich oil solvent 221 leaving reboiled Oil Absorber 205 may be pumped and fed to Solvent Regenerator 210. Solvent Regenerator 210 may comprise a trayed or packed tower, and may be operated at any suitable pressure, for example, approximately 30-35 barg. In some embodiments, refrigerated reflux condenser 223 may be operated at a temperature low enough such that the overhead stream 224 is condensed and collected in Solvent Regenerator reflux drum 225. The liquid C2-C4 stream 226 may be returned to Solvent Regenerator 210 as reflux through pump 228. Additionally, a portion of liquid C2-C4 stream 226 may be withdrawn under flow control as liquid C2-C4 product stream 229 and send to C2-C3 Splitter tower 215.

Solvent Regenerator reboiler (not shown) may be serviced by fired heater 312 which may be operated at any suitable temperature, for example, approximately 260° C. to 280° C. In the illustrated embodiment, bottoms stream 313 of Solvent Regenerator 210 is pumped to any suitable pressure, for example, 45 barg or greater, heated in a fired heater 312 and split into stream 314 that flashes to Solvent Regenerator 210 as vapor and lean absorption oil stream 310 at any suitable temperature, for example, approximately 260° C. to 270° C., which is circulated to heat reboilers 222 and 240 in series.

C2-C3 Splitter 215 may comprise a trayed or packed tower, which may be operated at any suitable pressure, for example, approximately 29-34 barg. In some embodiments, refrigerated reflux condenser 234 may be operated at a temperature such that the overhead stream 235 may be condensed. The liquid ethylene-ethane stream may be collected in the regenerator reflux drum 236 returned to the column via pump 237 as reflux. A portion of the condensed overhead stream may be withdrawn as product dilute ethylene-ethane stream 238 may be withdrawn and sent to the dilute ethylene product storage facility or other further processing steps.

C2-C3 Splitter 215 may be designed for the product dilute ethylene-ethane stream 238 to comprise a propylene content less than about 1.0 mppm; and the C2-C3 Splitter 215 bottoms stream 239 to comprise the C3's-C4's with ethane content to be less than approximately 5.0 mppm.

The C2-C3 Splitter reboiler 240 may be operated at about 70° C. to about 90° C. Bottoms stream 239 drawn from C2-C3 Splitter 215 may comprise substantially all the C3's-C4's in Clean ROG stream 208. Bottoms stream 239 may be sent for processing in the FCC unit debutanizer tower for recovery.

In some embodiments, both the reboiler 222 which services reboiled Oil Absorber 205 and Reboiler 240 which services C2-C3 Splitter 215 may be heat integrated from lean absorption oil stream 310 leaving fired heater 312. Lean absorption oil stream 310 may be cooled successively, for example, to approximately 150° C. in reboiler 222 and then further cooled, for example, to approximately 140° C., in reboiler 240. Lean absorption oil stream 310 may further cooled in successive exchangers, for example, to approximately 55° C. by air cooling in air cooler 315, then further cooled, for example, to approximately 47° C., by water cooling in exchanger 316, followed by cooling, for example, to approximately −2° C., by warm propylene refrigerant in exchanger 317 and then further cooling, for example, to approximately −35° C., by cold propylene refrigerant in exchanger 318.

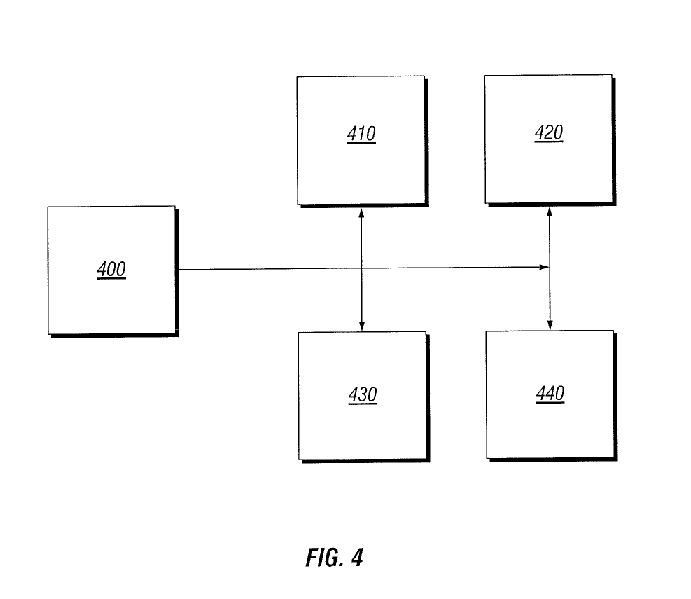

FIG. 4 illustrates several processes that further process product dilute ethylene stream 238 in accordance with present embodiments. Dilute ethylene production from ROG provides a platform for any refiner to enable direct integration within the refinery battery limits with other refinery units. ROG unit 400 may encompass any of the systems described herein, such as system 200 and system 300. Some non-limiting examples of further processing may be on-purpose propylene production in hydrocarbon metathesis unit 410. Propylene by metathesis chemistry may comprise catalytic conversion of dilute ethylene with butenes. Some examples of such units may be offered through CBI/Lummus and others. Another further processing step may comprise production of ethylbenzene in ethylbenzene unit 420. Alkylation of dilute ethylene with benzene to produce ethylbenzene may have various uses such as for motor gasoline blendstock or direct market sales as a merchantable product. Some examples of ethylbenzene units may be offered through Badger Licensing LLC and others. Another further processing step may comprise direct catalytic dimerization of dilute ethylene to butenes in alkylation unit 430. Dimerization may produce additional alkylate for use within the refinery or may be directly as merchantable butene-1 as a desired co-monomer with ethylene for linear low density polyethylene (LLDPE). Some examples of alkylation units may be offered through AXENS SA and others. Another further processing step may be direct chlorination of dilute ethylene for the production of ethylene dichloride (EDC) in chlorination unit 440. Ethylene dichloride may have many industrial uses including as the starting material for vinyl chloride monomer (VCM) for polyvinyl chloride (PVC) plastics. Examples of chlorination units may be OxyVinyls LLC and others.

It should be understood that the systems and methods are described in terms of “comprising,” “containing,” or “including” various components or steps, the compositions and methods can also “consist essentially of or “consist of” the various components and steps. Moreover, the indefinite articles “a” or “an,” as used in the claims, are defined herein to mean one or more than one of the element that it introduces.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present disclosure is well adapted to attain the ends and advantages mentioned as well as those that are inherent therein. The particular examples disclosed above are illustrative only, as the present invention may be modified and practiced in different but equivalent manners apparent to those skilled in the art having the benefit of the teachings herein. Although individual examples are discussed, the invention covers all combinations of all those examples. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative examples disclosed above may be altered or modified and all such variations are considered within the scope and spirit of the present invention. If there is any conflict in the usages of a word or term in this specification and one or more patent(s) or other documents that may be incorporated herein by reference, the definitions that are consistent with this specification should be adopted.

1. A method comprising:
introducing a refinery off gas stream into an oil absorber wherein the refinery off gas stream comprises H₂, N₂, O₂, methane, ethane, ethylene, propane, propylene, and C₄+;
introducing a solvent into the oil absorber;
counter-currently contacting the refinery off gas stream and the solvent in the oil absorber;
generating an absorber overhead stream comprising H₂, N₂, O₂, and methane;
generating an absorber bottoms stream comprising the solvent wherein ethane, ethylene, propane, propylene, and C₄+ are dissolved in the solvent;
introducing the absorber bottoms stream into a solvent regenerator and generating an overhead stream comprising ethane, ethylene, propane, propylene, and C₄+; and
introducing the overhead stream into a C₂-C₃ splitter that generates a dilute ethylene product stream and a bottoms product stream, wherein the dilute ethylene product stream comprises ethylene and ethane, and wherein the bottoms product stream comprises propane, propylene, and C₄+.

2. The method of claim 1 wherein the dilute ethylene product stream comprises less than 1 mppm each of H₂, N₂, O₂, and methane.

3. The method of claim 1 wherein the solvent comprises at least one solvent selected from the group consisting of cyclohexene, cyclohexane, hexane, hexene, heptane, octane, gasoline, kerosene and aromatic distillate.

4. The method of claim 1 wherein the absorber bottoms stream contains about 70% to about 82% of the mass of the ethylene from the refinery off gas stream.

5. The method of claim 1 further comprising:
sending the absorber overhead stream to a pressure swing absorption unit and generating an H₂ product stream wherein the H₂ product stream is greater than 99.99% H₂ by weight.

6. The method of claim 1 further comprising at least one of the following steps: sending the dilute ethylene product stream combined with butylenes to a metathesis unit to produce propylene; sending the dilute ethylene product stream to an alkylation unit to produce ethylbenzene by benzene alkylation; sending the dilute ethylene product stream to a dimerization unit to produce butenes; sending the dilute ethylene product stream to chlorination unit to produce ethylene dichloride, or a combination thereof.

7. A method comprising:
cracking a hydrocarbon feedstock in a fluidized catalytic cracking unit to produce a refinery off gas stream wherein the refinery off gas stream comprises H₂, N₂, O₂, methane, ethane, ethylene, propane, propylene, and C₄+;
introducing the refinery off gas stream into an oil absorber;
introducing a lean solvent into the oil absorber;
counter-currently contacting the refinery off gas stream and the lean solvent in the oil absorber and absorbing at least a portion of the ethane, ethylene, propane, propylene, and C₄+ into the lean solvent to generate a spent solvent and an overhead stream comprising H₂, N₂, O₂, and methane;
drawing a bottoms stream comprising the spent solvent with absorbed ethane, ethylene, propane, propylene, and C₄+ and introducing the bottoms stream into a solvent regenerator;
removing at least a portion of the absorbed ethane, ethylene, propane, propylene, and C₄+ from the spent solvent and forming an overhead stream comprising the removed ethane, ethylene, propane, propylene, and C₄+; and
separating the overhead stream in a distillation column to generate a dilute ethylene product stream comprising ethane and ethylene wherein the dilute ethylene stream contains less than 1 mppm each of H₂, N₂, O₂, and methane and a bottoms product stream comprising propane, propylene, and C₄+.

8. The method of claim 7 wherein the solvent comprises a pure component solvent selected from the group consisting of cyclohexene, cyclohexane, hexane, hexene, heptane, octane, gasoline, kerosene and aromatic distillate or a mixture of components from the selected group.

9. The method of claim 7 further comprising crossing the overhead stream and the refinery off gas stream in a pre-cooler heat exchanger prior to the step of introducing the refinery off gas stream into an oil absorber.

10. The method of claim 9 wherein the overhead stream is further sent to a pressure swing adsorption unit.

11. The method of claim 10 wherein the pressure swing adsorption unit generates a H₂ product stream wherein the H₂ product stream is greater than 99.99% H₂ by weight.

12. The method of claim 7 wherein the step of removing further comprises generating the lean solvent from the spent solvent.

13. The method of claim 12 further comprising:
heating the lean solvent in a fired heater;
drawing a first reboil stream from the oil absorber and crossing the lean solvent and first reboil stream in a first heat exchanger; and
drawing a second reboil stream from the distillation column and crossing the lean solvent and second reboil stream in a second heat exchanger.

14. The method of claim 13 further comprising cooling the lean solvent to about −35° C. to −40° C.

15. The method of claim 14 wherein the step of cooling comprises:
cooling the lean solvent in an air cooler;
cooling the lean solvent in a cooling water exchanger; and
cooling the lean solvent in a refrigerant exchanger.

16. The method of claim 15 wherein the refrigerant exchanger comprises a first propylene refrigerant exchanger operating at about 2° C. to about −5° C. and a second propylene refrigerant exchanger operating at about −35° C. to about −40° C.

17. A system comprising:
an oil absorber coupled to the fluidized catalytic cracking unit to receive refinery off gas from a fluidized catalytic cracking unit;
a solvent regenerator coupled to the oil absorber to receive an output of the oil absorber; and
a C₂-C₃ splitter coupled to the solvent regenerator to receive an output of the solvent regenerator.

18. The system of claim 17 wherein the fluidized catalytic cracking unit is configured to output a refinery off gas comprising H₂, N₂, O₂, methane, ethane, ethylene, propane, propylene, and C₄+ and wherein the oil absorber is configured to counter currently contact a solvent and the refinery off gas to absorb at least a portion of the ethane, ethylene, propane, propylene, and C₄+ into the solvent and output the solvent to the solvent regenerator.

19. The system of claim 18 wherein the solvent regenerator is configured to remove at least a portion of the ethane, ethylene, propane, propylene, and C₄+ from the solvent and output a gas stream comprising the ethane, ethylene, propane, propylene, and C₄+ to the C₂-C₃ splitter.

20. The system of claim 19 wherein the C₂-C₃ splitter is configured to generate a product dilute ethylene stream comprising ethylene and ethane and a bottoms stream comprising propane, propylene, and C₄+.
